(19) United States
(12) Patent Application Publication
(10) Pub. No.: US 2026/0240508 A1
(43) Pub. Date: Aug. 20, 2026

(54) X-RAY GENERATION DEVICE AND DISCHARGE LOCATION IDENTIFICATION METHOD

(71) Applicant: FUJIFILM Corporation, Tokyo (JP)

(72) Inventor: Shotaro SHINDO, Tokyo (JP)

(73) Assignee: FUJIFILM Corporation, Tokyo (JP)

(21) Appl. No.: 19/537,603

(22) Filed: Feb. 12, 2026

(30) Foreign Application Priority Data
Feb. 19, 2025 (JP) 2025-025298

(51) Int. Cl. A61B 6/03

(52) U.S. Cl. CPC A61B 6/032

(57) ABSTRACT

An X-ray generation device according to the technology of the present disclosure includes a high-voltage generator that generates a high voltage, an X-ray tube that generates X-rays in response to the high voltage supplied from the high-voltage generator, a discharge phenomenon detection circuit that detects an occurrence of a discharge phenomenon, a vibration sensor that is attached to the high-voltage generator, and a determination circuit that determines whether the discharge phenomenon occurs in the X-ray tube or the high-voltage generator based on detection results of the discharge phenomenon detection circuit and the vibration sensor.

### CROSS-REFERENCE TO RELATED APPLICATION

The present application claims priority under 35 U.S.C. § 119 to Japanese Patent Application No. 2025-025298, filed on Feb. 19, 2025. The above application is hereby expressly incorporated by reference, in its entirety, into the present application.

### BACKGROUND

### 1. Technical Field

The present disclosure relates to an X-ray generation device and a discharge location identification method.

### 2. Description of the Related Art

An image diagnostic apparatus using X-rays, such as a computed tomography (CT) apparatus, irradiates a subject with X-rays generated by an X-ray generation device and detects and images an X-ray amount transmitted through the subject. The X-ray generation device is configured to include a high-voltage generation device that generates a direct current high voltage, and an X-ray tube that generates X-rays by the high voltage generated by the high-voltage generation device. The X-ray tube includes a container in which an inside is maintained in a vacuum, and a cathode and an anode that are provided to face each other in the container. The cathode releases thermal electrons toward the anode by applying the high voltage. The anode generates X-rays by collision with the thermal electrons released from the cathode.

The X-ray tube is required to be periodically replaced because a load such as the high voltage is applied, but since the X-ray tube is generally very expensive, it is desirable to use the X-ray tube until the life is completely exhausted and then replace the X-ray tube. For this reason, a technology that enables detection of a deterioration state of the X-ray tube is known (for example, refer to JP2017-224481A and JP2011-045626A).

### SUMMARY

However, the X-ray tube may fail during use. One of the failure causes is a discharge phenomenon. For example, in a case where the vacuum degree in the X-ray tube is deteriorated, an arc discharge occurs between the cathode and the anode, so that a predetermined high voltage is not applied between the cathode and the anode, and a problem occurs in X-ray generation. In addition, the discharge phenomenon is not limited to the X-ray tube, and may occur in the high-voltage generator. Therefore, in a case where a failure occurs due to the discharge phenomenon, in order to replace only the X-ray tube or the high-voltage generator in which the discharge phenomenon occurs, it is necessary to identify whether the discharge phenomenon occurs in the X-ray tube or the high-voltage generator.

JP2017-224481A and JP2011-045626A describe detection of the discharge phenomenon generated in the X-ray tube, but do not describe identification of whether the discharge phenomenon occurs in the X-ray tube or the high-voltage generator.

Therefore, an object of the technology according to the present disclosure is to provide an X-ray generation device and a discharge location identification method that enable identification of whether the discharge phenomenon occurs in the X-ray tube or the high-voltage generator.

An X-ray generation device according to the technology of the present disclosure includes a high-voltage generator that generates a high voltage, an X-ray tube that generates X-rays in response to the high voltage supplied from the high-voltage generator, a discharge phenomenon detection circuit that detects an occurrence of a discharge phenomenon, a vibration sensor that is attached to the high-voltage generator, and a determination circuit that determines whether the discharge phenomenon occurs in the X-ray tube or the high-voltage generator based on detection results of the discharge phenomenon detection circuit and the vibration sensor.

It is preferable that the determination circuit determines that the discharge phenomenon occurs in the high-voltage generator in a case where the discharge phenomenon detection circuit detects the occurrence of the discharge phenomenon and the vibration sensor detects a discharge vibration caused by the discharge phenomenon.

It is preferable that the determination circuit determines that the discharge phenomenon occurs in the X-ray tube in a case where the discharge phenomenon detection circuit detects the occurrence of the discharge phenomenon and the vibration sensor does not detect the discharge vibration caused by the discharge phenomenon.

It is preferable that the determination circuit determines that the discharge vibration is detected in a case where an amplitude or a peak value of a vibration detected by the vibration sensor is equal to or greater than a threshold value.

It is preferable that the determination circuit determines that the discharge vibration is detected in a case where a waveform of a vibration detected by the vibration sensor matches a discharge waveform stored in advance.

It is preferable that the discharge phenomenon detection circuit detects the discharge phenomenon based on a change in at least one of a tube current or a tube voltage of the X-ray tube.

It is preferable that the high-voltage generator and the X-ray tube use a neutral point grounding method.

It is also preferable that the high-voltage generator and the X-ray tube use an anode grounding method.

It is preferable that the vibration sensor is a displacement sensor or an acceleration sensor.

A discharge location identification method according to the technology of the present disclosure is a discharge location identification method for an X-ray generation device including a high-voltage generator that generates a high voltage, an X-ray tube that generates X-rays in response to the high voltage supplied from the high-voltage generator, a discharge phenomenon detection circuit that detects an occurrence of a discharge phenomenon, and a vibration sensor that is attached to the high-voltage generator, the method comprising: determining whether the discharge phenomenon occurs in the X-ray tube or the high-voltage generator based on detection results of the discharge phenomenon detection circuit and the vibration sensor.

According to the technology of the present disclosure, it is possible to provide an X-ray generation device and a discharge location identification method that enable identification of whether the discharge phenomenon occurs in the X-ray tube or the high-voltage generator.

### BRIEF DESCRIPTION OF THE DRAWINGS

Exemplary embodiments according to the technique of the present disclosure will be described in detail based on the following figures, wherein:

FIG. 1 is a diagram schematically showing a configuration of a CT apparatus,

FIG. 2 is a diagram showing a functional configuration of an X-ray generation device,

FIG. 3 is a diagram schematically showing a circuit configuration of a high-voltage generation device and an X-ray tube,

FIG. 4 is a diagram showing an example of a flow of determination processing by a determination unit,

FIG. 5 is a diagram showing details of detection determination processing of a discharge vibration,

FIG. 6 is a diagram showing an example of a vibration waveform,

FIG. 7 is a diagram showing an example of the vibration waveform, and

FIG. 8 is a diagram schematically showing a circuit configuration of a high-voltage generation device and an X-ray tube according to a modification example.

### DETAILED DESCRIPTION

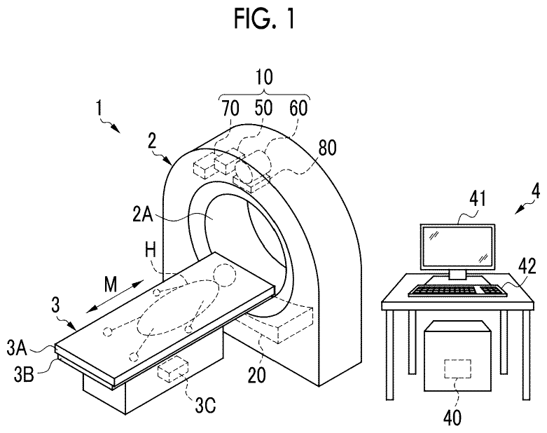

FIG. 1 schematically shows the configuration of a CT apparatus 1. The CT apparatus 1 is composed of a gantry 2, a bed 3, and a console 4.

The gantry 2 has an opening 2A in the center through which a part of the bed 3 is inserted. An X-ray generation device 10 that generates X-rays to be emitted to a subject H and a detector 20 that detects the X-rays transmitted through the subject H and generates a radiation image are provided inside the gantry 2.

The bed 3 has a top plate 3A on which the subject H is placed, a base portion 3B that supports the top plate 3A, and a driving unit 3C that moves the top plate 3A back and forth in the direction of arrow M, and is configured to allow the subject H to be moved. The top plate 3A can be slid in the direction of arrow M relative to the base portion 3B by the driving unit 3C. In a case where the subject H is imaged, the top plate 3A slides such that the top plate 3A is inserted into the opening 2A of the gantry 2. Accordingly, the subject H is transported into the opening 2A.

The console 4 is a computer including a processor 40 such as a central processing unit (CPU), a display 41 such as a liquid-crystal display, and an input device 42 such as a keyboard and a mouse. The console 4 can perform a setting operation of X-ray irradiation conditions or the like and can display the set X-ray irradiation conditions or the like.

The X-ray generation device 10 includes a high-voltage generation device 50, an X-ray tube 60, an X-ray control device 70, and a stop 80. The high-voltage generation device 50 generates a direct current high voltage. The X-ray tube 60 generates X-rays in response to the high voltage supplied from the high-voltage generation device 50 and emits the generated X-rays to the subject H. The stop 80 shapes the X-rays generated by the X-ray tube 60 into a cone beam having a predetermined fan angle and cone angle.

The X-ray control device 70 performs control such that the tube current and the tube voltage of the X-ray tube 60 match set values set on the console 4. In addition, the X-ray control device 70 detects the discharge phenomenon described below and identifies a discharge location.

The detector 20 includes a plurality of X-ray detection elements. The detector 20 detects data indicating the intensity distribution of X-rays transmitted through the subject H using a plurality of X-ray detection elements, and outputs the data. For example, the detector 20 is a two-dimensional X-ray detector in which a plurality of X-ray detection elements are disposed in two mutually orthogonal directions (that is, a slice direction and a channel direction). The detector 20 can image a three-dimensional imaging region having a width in the slice direction in one rotation scan.

The X-ray control device 70 and the detector 20 are configured to be rotatable along the annular shape of the gantry 2 while maintaining a mutually opposing positional relationship.

A stop driving unit (not shown) for driving the X-ray control device 70 and the stop 80 and a gantry driving unit (not shown) for driving the gantry 2 are controlled by the console 4. The console 4 also controls the driving unit 3C of the bed 3.

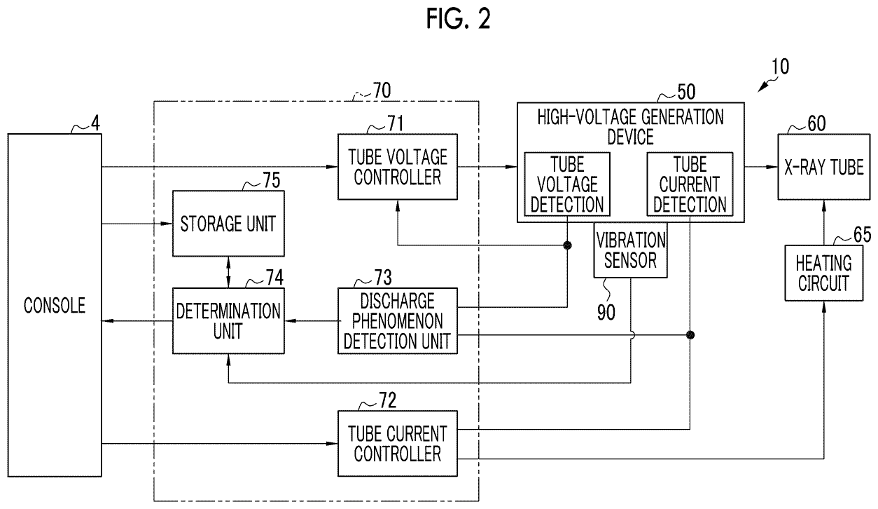

FIG. 2 shows a functional configuration of the X-ray generation device 10. A vibration sensor 90 that detects a vibration generated in the high-voltage generation device 50 is attached to the high-voltage generation device 50. For example, the vibration sensor 90 is attached to a case that accommodates a plurality of components of the high-voltage generation device 50. The vibration sensor 90 is a displacement sensor that detects a displacement amount of the high-voltage generation device 50 and outputs a detection value to the X-ray control device 70. The vibration sensor 90 may be an acceleration sensor. In this case, the displacement amount of the high-voltage generation device 50 can be calculated by integrating the acceleration, which is the detection value, twice. Note that the high-voltage generation device 50 corresponds to the high-voltage generator in the present disclosure.

The X-ray control device 70 functions as a tube voltage controller 71, a tube current controller 72, a discharge phenomenon detection unit 73, a determination unit 74, and a storage unit 75. For example, the X-ray control device 70 is configured by a processor such as a CPU, and the processor executes processing based on a program stored in the storage unit 75 to realize the various functions. Note that the determination unit 74 corresponds to the determination circuit in the present disclosure, and the discharge phenomenon detection unit 73 corresponds to the discharge phenomenon detection circuit in the present disclosure.

The tube voltage controller 71 controls the high voltage (that is, the tube voltage) applied to the X-ray tube 60 by the high-voltage generation device 50 based on a set value of the tube voltage set by the console 4. In addition, the detection value of the tube voltage is acquired from the high-voltage generation device 50, and feedback control is performed such that the detection value is the set value of the tube voltage.

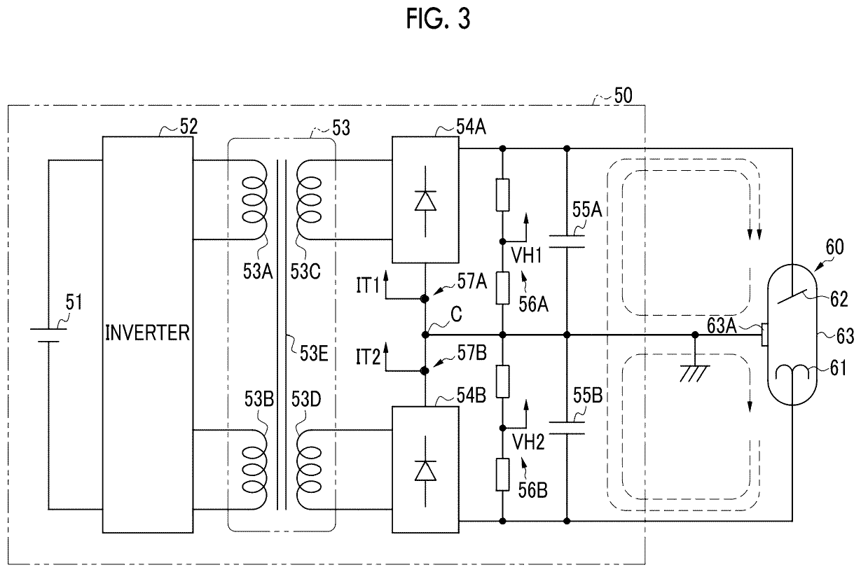

The tube current controller 72 controls the tube current by controlling a heating circuit 65 that heats a cathode 61 (refer to FIG. 3) of the X-ray tube 60 based on a set value of the tube current set by the console 4. In addition, the tube current controller 72 acquires the detection value of the tube current from the high-voltage generation device 50, and performs feedback control such that the detection value is the set value of the tube current.

As shown in FIG. 3, the X-ray tube 60 is configured of a container 63 in which an inside is maintained in a vacuum, and a cathode 61 and an anode 62 that are provided to face each other in the container 63. The anode 62 may be a rotating anode that rotates by a rotor.

The discharge phenomenon detection unit 73 is a detector that detects the discharge phenomenon generated in the X-ray generation device 10 in a case of a failure or the like. The discharge phenomenon occurs in a case where the X-ray generation device 10 generates X-rays, due to insulation breakdown caused by deterioration of the vacuum degree or the like. For example, in a case where the vacuum degree in the container 63 is deteriorated, an arc discharge occurs between the cathode 61 and the anode 62, so that a predetermined high voltage is not applied between the cathode 61 and the anode 62, and a problem occurs in X-ray generation. In addition, the discharge phenomenon is not limited to the X-ray tube 60, and may occur in the high-voltage generation device 50.

In the present embodiment, the discharge phenomenon detection unit 73 detects the occurrence of the discharge phenomenon based on a change in at least one of the tube current or the tube voltage. This is based on the fact that at least one of the tube current or the tube voltage changes abruptly in a case where the discharge phenomenon occurs, as compared with a case of normal operation. The discharge phenomenon detection unit 73 may detect the occurrence of the discharge phenomenon based on any one of the detection value of the tube current or the detection value of the tube voltage, or may detect the occurrence of the discharge phenomenon based on both of the detection value of the tube current and the detection value of the tube voltage.

The determination unit 74 determines whether the discharge phenomenon occurs in the X-ray tube 60 or the high-voltage generation device 50 based on the detection results of the discharge phenomenon detection unit 73 and the vibration sensor 90. Although the details will be described below, the determination unit 74 determines that the discharge phenomenon occurs in the high-voltage generation device 50 in a case where the discharge phenomenon detection unit 73 detects the occurrence of the discharge phenomenon and the vibration sensor 90 detects a vibration (hereinafter, referred to as a discharge vibration) caused by the discharge phenomenon. On the other hand, the determination unit 74 determines that the discharge phenomenon occurs in the X-ray tube 60 in a case where the discharge phenomenon detection unit 73 detects the occurrence of the discharge phenomenon and the vibration sensor 90 does not detect the discharge vibration.

The storage unit 75 is a memory such as a random access memory (RAM), and stores data such as a threshold value used in a case where the determination unit 74 performs the determination processing. In addition, the storage unit 75 stores the detection value of the vibration detected by the vibration sensor 90 as waveform data.

FIG. 3 schematically shows a circuit configuration of the high-voltage generation device 50 and the X-ray tube 60. The high-voltage generation device 50 and the X-ray tube 60 according to the present embodiment use a neutral point grounding method. In the X-ray tube 60, a metal portion 63A is provided in a part of the container 63, and the metal portion 63A is connected to a ground.

The high-voltage generation device 50 includes a direct current power source 51, an inverter 52, a high-voltage transformer 53, a first rectifier circuit 54A, a second rectifier circuit 54B, a first capacitor 55A, and a second capacitor 55B. The direct current power source 51 generates a direct current voltage. The inverter 52 converts the direct current voltage generated by the direct current power source 51 into an alternating current voltage having a predetermined frequency.

The high-voltage transformer 53 boosts the alternating current voltage generated by the inverter 52. The high-voltage transformer 53 includes two primary windings 53A and 53B, two secondary windings 53C and 53D, and a core 53E. The primary windings 53A and 53B are connected in parallel to an output of the inverter 52. The secondary windings 53C and 53D are disposed to face the primary windings 53A and 53B with the core 53E interposed therebetween.

The first rectifier circuit 54A is connected to the secondary winding 53C and converts the output voltage of the secondary winding 53C into a direct current first high voltage VH1. The second rectifier circuit 54B is connected to the secondary winding 53D and converts the output voltage of the secondary winding 53D into a direct current second high voltage VH2.

The first rectifier circuit 54A and the first capacitor 55A are connected in parallel between the anode 62 of the X-ray tube 60 and the ground. The second rectifier circuit 54B and the second capacitor 55B are connected in parallel between the cathode 61 of the X-ray tube 60 and the ground. That is, the high-voltage generation device 50 uses a neutral point grounding method in which a negative side of a direct current output terminal of the first rectifier circuit 54A and a positive side of a direct current output terminal of the second rectifier circuit 54B are connected to each other, and a connection point C is grounded.

The first rectifier circuit 54A applies the first high voltage VH1 between the anode 62 and the ground. The second rectifier circuit 54B applies the second high voltage VH2 between the cathode 61 and the ground. A sum of the first high voltage VH1 and the second high voltage VH2 corresponds to the tube voltage.

The high-voltage generation device 50 is provided with a first voltage detection unit 56A that detects the first high voltage VH1 and a second voltage detection unit 56B that detects the second high voltage VH2. The first voltage detection unit 56A includes a detection resistor and is connected between the anode 62 and the ground. The second voltage detection unit 56B includes a detection resistor and is connected between the cathode 61 and the ground. The detection values of the first high voltage VH1 and the second high voltage VH2 are input to the tube voltage controller 71 and the discharge phenomenon detection unit 73 as the detection value of the tube voltage.

In addition, the high-voltage generation device 50 is provided with a first current detection unit 57A that detects a first current IT1 flowing on the anode 62 side and a second current detection unit 57B that detects a second current IT2 flowing on the cathode 61 side. The first current detection unit 57A includes a detection resistor and is disposed between the negative side of the direct current output terminal of the first rectifier circuit 54A and the ground. The second current detection unit 57B includes a detection resistor and is disposed between the positive side of the direct current output terminal of the second rectifier circuit 54B and the ground. A sum of the first current IT1 and the second current IT2 corresponds to the tube current. The detection values of the first current IT1 and the second current IT2 are input to the tube current controller 72 and the discharge phenomenon detection unit 73 as the detection value of the tube current.

A plurality of broken lines shown in FIG. 3 indicate a path of a current (hereinafter, referred to as a discharge current) flowing due to the discharge phenomenon generated in the X-ray tube 60. In the high-voltage generation device 50 of the neutral point grounding method, the first current detection unit 57A and the second current detection unit 57B cannot be disposed on the path through which the discharge current flows due to a relationship in which the first current detection unit 57A and the second current detection unit 57B need to be disposed on the low-voltage side.

Therefore, in the high-voltage generation device 50 of the neutral point grounding method, since the first current detection unit 57A and the second current detection unit 57B cannot directly detect the discharge current, the discharge phenomenon detection unit 73 detects the occurrence of the discharge phenomenon based on a change in at least one of the tube current or the tube voltage generated due to the discharge phenomenon. For example, after the inverter 52 stops the operation, a change in at least one of the tube current or the tube voltage is different between a case of normal operation in which the discharge phenomenon does not occur and a case of abnormal operation in which the discharge phenomenon occurs, so that the discharge phenomenon detection unit 73 detects the occurrence of the discharge phenomenon based on the difference.

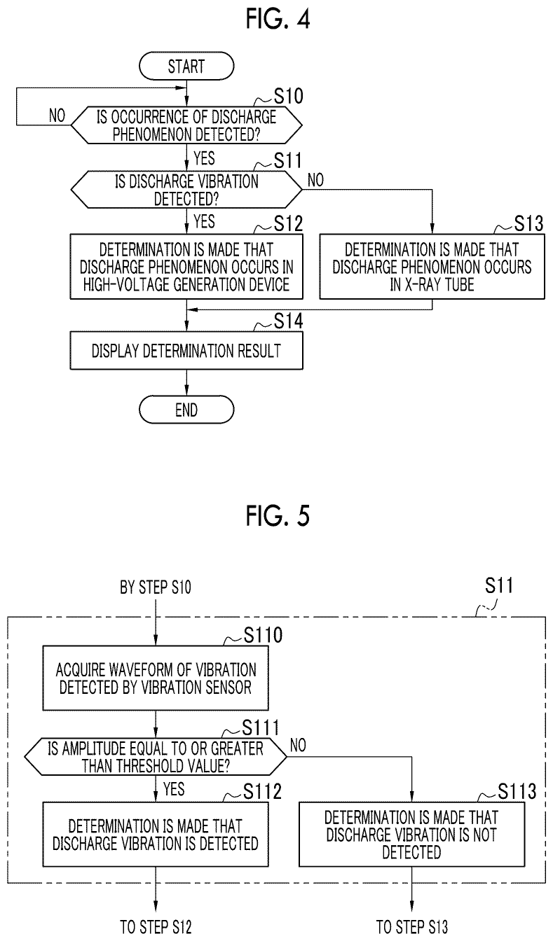

FIG. 4 shows an example of a flow of the determination processing by the determination unit 74. The determination unit 74 determines whether the discharge phenomenon detection unit 73 detects the occurrence of the discharge phenomenon (step S10). In a case where the determination is negative (step S10: NO), the determination unit 74 repeats the determination. In a case where the determination is affirmative (step S10: YES), the determination unit 74 determines whether the discharge vibration is detected based on the detection value of the vibration sensor 90 (step S11).

In a case where the determination is affirmative (step S11: YES), the determination unit 74 determines that the discharge phenomenon occurs in the high-voltage generation device 50 (step S12). This is based on the fact that, in a case where the discharge phenomenon occurs in the high-voltage generation device 50, the discharge vibration is detected by the vibration sensor 90.

On the other hand, in a case where the determination is negative (step S11: NO), the determination unit 74 determines that the discharge phenomenon occurs in the X-ray tube 60 (step S13). This is based on the fact that, in a case where the discharge phenomenon occurs in the X-ray tube 60, the discharge vibration is not detected by the vibration sensor 90.

Thereafter, the console 4 displays the determination result of the determination unit 74 on the display 41 (step S14).

FIG. 5 shows details of the detection determination processing of the discharge vibration (step S11). After the determination is affirmative in step S10, the determination unit 74 acquires a waveform (hereinafter, referred to as a vibration waveform) of the vibration detected by the vibration sensor 90 in a predetermined period (step S110). Next, the determination unit 74 determines whether an amplitude of the vibration is equal to or greater than a threshold value based on the acquired vibration waveform (step S111). In a case where the determination is affirmative (step S111: YES), the determination unit 74 determines that the discharge vibration is detected (step S112). On the other hand, in a case where the determination is negative (step S111: NO), the determination unit 74 determines that the discharge vibration is not detected (step S113).

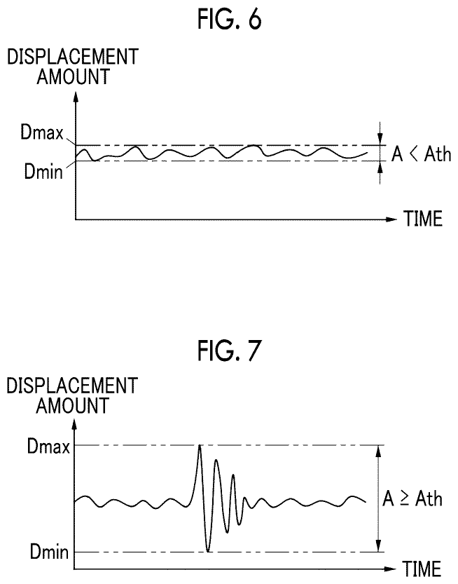

FIGS. 6 and 7 show an example of the vibration waveform. In FIGS. 6 and 7, an amplitude A of the vibration waveform is defined by a difference value between a maximum value Dₘₐₓ and a minimum value Dₘᵢₙ of the vibration waveform.

FIG. 6 shows an example of the vibration waveform in a case where the discharge vibration is not detected. As shown in FIG. 6, in a case where the amplitude A of the vibration waveform is smaller than a threshold value Aₜₕ, it is determined that the discharge vibration is not detected. Such a vibration waveform is, for example, a minute vibration caused by an operation of the CT apparatus 1 such as the rotation of the gantry 2.

FIG. 7 shows an example of the vibration waveform in a case where the discharge vibration is detected. As shown in FIG. 7, in a case where the amplitude A of the vibration waveform is equal to or greater than a threshold value Aₜₕ, it is determined that the discharge vibration is detected. As described above, by comparing the amplitude A of the vibration waveform with the threshold value Aₜₕ to perform the determination, the minute vibration can be prevented from being erroneously detected as the discharge vibration, and the discharge vibration can be accurately detected.

The determination unit 74 may compare a peak value (for example, a maximum peak value) of the vibration waveform with the threshold value, and determine that the discharge vibration is detected in a case where the peak value is equal to or greater than the threshold value, instead of the amplitude of the vibration waveform.

In addition, the determination unit 74 may read out the discharge waveform stored in advance in the storage unit 75, and determine that the discharge vibration is detected in a case where the vibration waveform matches the discharge waveform stored in advance. The match means that a match degree obtained by a correlation operation or the like is equal to or greater than a certain value.

As described above, according to the present embodiment, in a case where the discharge phenomenon occurs due to a failure or the like of the X-ray generation device 10, it is possible to identify whether the discharge phenomenon occurs in the X-ray tube 60 or the high-voltage generation device 50. As described above, according to the present embodiment, the discharge location can be easily identified, and only the X-ray tube 60 or the high-voltage generation device 50 in which the discharge phenomenon occurs can be quickly replaced.

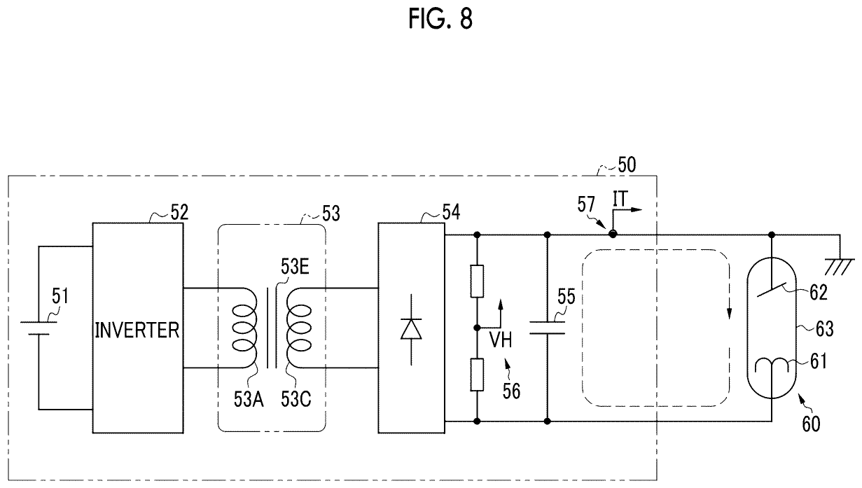

Next, a modification example of the X-ray tube 60 and the high-voltage generation device 50 will be described. FIG. 8 schematically shows a circuit configuration of the high-voltage generation device 50 and the X-ray tube 60 according to the modification example. The high-voltage generation device 50 and the X-ray tube 60 according to the present modification example use an anode grounding method. The X-ray tube 60 according to the present modification example has the same configuration as that of the above-described embodiment, except that the anode 62 is connected to the ground instead of the metal portion 63A (refer to FIG. 3).

The high-voltage generation device 50 according to the present modification example includes a direct current power source 51, an inverter 52, a high-voltage transformer 53, a rectifier circuit 54, and a capacitor 55. The direct current power source 51 and the inverter 52 have the same configuration as that of the above-described embodiment.

In the present modification example, the high-voltage transformer 53 includes a primary winding 53A, a secondary winding 53C, and a core 53E. The primary winding 53A is connected to an output of the inverter 52. The secondary winding 53C is disposed to face the primary winding 53A with the core 53E interposed therebetween.

The rectifier circuit 54 is connected to the secondary winding 53C and converts the output voltage of the secondary winding 53C into a direct current high voltage VH.

The rectifier circuit 54 and the capacitor 55 are connected in parallel between the anode 62 of the X-ray tube 60 and the ground. That is, the high-voltage generation device 50 uses an anode grounding method in which the positive side of the direct current output terminal of the rectifier circuit 54 is grounded.

The rectifier circuit 54 applies the high voltage VH between the cathode 61 and the anode 62 of the X-ray tube 60. In the present modification example, the high voltage VH corresponds to the tube voltage.

The high-voltage generation device 50 is provided with a voltage detection unit 56 that detects the high voltage VH. The voltage detection unit 56 includes a detection resistor and is connected between the cathode 61 and the anode 62 of the X-ray tube 60. The detection value of the high voltage VH is input to the tube voltage controller 71 and the discharge phenomenon detection unit 73 as the detection value of the tube voltage.

In addition, the high-voltage generation device 50 is provided with a current detection unit 57 that detects a current IT flowing on the anode 62 side. The current detection unit 57 includes a detection resistor and is connected between the positive side of the direct current output terminal of the rectifier circuit 54 and the anode 62. The detection value of the current IT is input to the tube current controller 72 and the discharge phenomenon detection unit 73 as the detection value of the tube current.

A broken line shown in FIG. 8 indicates a path of the discharge current generated in the X-ray tube 60. In the present modification example, the current detection unit 57 can be disposed on the path through which the discharge current flows. Therefore, in the present modification example, since the discharge current can be detected by the current detection unit 57, the occurrence of the discharge phenomenon in the X-ray tube 60 can be directly detected.

Even in the present modification example, in a case where the discharge phenomenon occurs due to a failure or the like of the X-ray generation device 10, it is possible to identify whether the discharge phenomenon occurs in the X-ray tube 60 or the high-voltage generation device 50. As described above, according to the present modification example, the discharge location can be easily identified, and only the X-ray tube 60 or the high-voltage generation device 50 in which the discharge phenomenon occurs can be quickly replaced.

In the above-described embodiment, the technology of the present disclosure has been described using the X-ray generation device 10 mounted on the CT apparatus 1 as an example, but the technology of the present disclosure can also be applied to an X-ray generation device mounted on an X-ray imaging apparatus other than the CT apparatus.

In the above-described embodiment, each processing executed by the X-ray control device 70 is executed by any computer. Moreover, any computer may execute these processes by a processor as hardware, a program as software, or a combination thereof. In such a case, the processor is configured to execute various types of processing in the above-described embodiment in cooperation with the program, and may function as each unit or each means in the above-described embodiment. Further, the execution order of the processing by the processor is not limited to the above-described order and may be changed as appropriate. Any computer may be a general-purpose computer, a computer for a specific application, a workstation, or another system capable of executing each process.

The processor may be composed of one or a plurality of pieces of hardware, and types of hardware are not limited. The processor may be configured by, for example, a (CPU), a micro processing unit (MPU), a programmable logic device such as a field programmable gate array (FPGA), a dedicated circuit for executing specific processing, such as an application specific integrated circuit (ASIC), or hardware such as a graphic processing unit (GPU) or a neural processing unit (NPU). Types of hardware may be a combination of different types of hardware. In a case where the plurality of types of hardware are configured to execute one or a plurality of types of processing of a certain processor, the plurality of types of hardware may exist in devices physically separated from each other or may exist in the same device. Further, in any of the embodiments, the order of each processing performed by the processor is not limited to the above-described order, and may be changed as appropriate. The hardware is configured by an electric circuit (circuitry) in which circuit elements, such as semiconductor elements, are combined, or the like.

Further, the program may be software, such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, an argument, a parameter, or contents of a memory.

Further, in the above-described embodiments, the aspect has been described in which a program is stored in the storage unit 75 in advance. However, the present disclosure is not limited thereto. The program may be provided in a form of being recorded on a recording medium, such as a compact disc read only memory (CD-ROM), a digital versatile disc read only memory (DVD-ROM), or a universal serial bus (USB) memory. In addition, the program may be downloaded from an external device via a network.

The technology of the present disclosure extends to any program products. The program products include products in any aspect for providing a program. For example, the program product includes a program provided through a network such as the Internet, and non-transitory computer-readable recording media such as a CD-ROM, a DVD, and a USB memory in which the program is stored.

It is possible to understand the technology according to the following supplementary notes, based on the above description.

### Supplementary Note 1

An X-ray generation device comprising:
a high-voltage generation device that generates a high voltage;
an X-ray tube that generates X-rays in response to the high voltage supplied from the high-voltage generation device;
a discharge phenomenon detection unit that detects an occurrence of a discharge phenomenon;
a vibration sensor that is attached to the high-voltage generation device; and
a determination unit that determines whether the discharge phenomenon occurs in the X-ray tube or the high-voltage generation device based on detection results of the discharge phenomenon detection unit and the vibration sensor.

### Supplementary Note 2

The X-ray generation device according to supplementary note 1,
wherein the determination unit determines that the discharge phenomenon occurs in the high-voltage generation device in a case where the discharge phenomenon detection unit detects the occurrence of the discharge phenomenon and the vibration sensor detects a discharge vibration caused by the discharge phenomenon.

### Supplementary Note 3

The X-ray generation device according to supplementary note 1 or 2,
wherein the determination unit determines that the discharge phenomenon occurs in the X-ray tube in a case where the discharge phenomenon detection unit detects the occurrence of the discharge phenomenon and the vibration sensor does not detect the discharge vibration caused by the discharge phenomenon.

### Supplementary Note 4

The X-ray generation device according to any one of supplementary notes 1 to 3,
wherein the determination unit determines that the discharge vibration is detected in a case where an amplitude or a peak value of a vibration detected by the vibration sensor is equal to or greater than a threshold value.

### Supplementary Note 5

The X-ray generation device according to any one of supplementary notes 1 to 3,
wherein the determination unit determines that the discharge vibration is detected in a case where a waveform of a vibration detected by the vibration sensor matches a discharge waveform stored in advance.

### Supplementary Note 6

The X-ray generation device according to any one of supplementary notes 1 to 5,
wherein the discharge phenomenon detection unit detects the discharge phenomenon based on a change in at least one of a tube current or a tube voltage of the X-ray tube.

### Supplementary Note 7

The X-ray generation device according to any one of supplementary notes 1 to 6,
wherein the high-voltage generation device and the X-ray tube use a neutral point grounding method.

### Supplementary Note 8

The X-ray generation device according to any one of supplementary notes 1 to 6,
wherein the high-voltage generation device and the X-ray tube use an anode grounding method.

### Supplementary Note 9

The X-ray generation device according to any one of supplementary notes 1 to 8,
wherein the vibration sensor is a displacement sensor or an acceleration sensor.

Explanation of References

What is claimed is:

1. An X-ray generation device comprising:
a high-voltage generator that generates a high voltage;
an X-ray tube that generates X-rays in response to the high voltage supplied from the high-voltage generator;
a discharge phenomenon detection circuit that detects an occurrence of a discharge phenomenon;
a vibration sensor that is attached to the high-voltage generator; and
a determination circuit that determines whether the discharge phenomenon occurs in the X-ray tube or the high-voltage generator based on detection results of the discharge phenomenon detection circuit and the vibration sensor.

2. The X-ray generation device according to claim 1,
wherein the determination circuit determines that the discharge phenomenon occurs in the high-voltage generator in a case where the discharge phenomenon detection circuit detects the occurrence of the discharge phenomenon and the vibration sensor detects a discharge vibration caused by the discharge phenomenon.

3. The X-ray generation device according to claim 2,
wherein the determination circuit determines that the discharge phenomenon occurs in the X-ray tube in a case where the discharge phenomenon detection circuit detects the occurrence of the discharge phenomenon and the vibration sensor does not detect the discharge vibration caused by the discharge phenomenon.

4. The X-ray generation device according to claim 3,
wherein the determination circuit determines that the discharge vibration is detected in a case where an amplitude or a peak value of a vibration detected by the vibration sensor is equal to or greater than a threshold value.

5. The X-ray generation device according to claim 3,
wherein the determination circuit determines that the discharge vibration is detected in a case where a waveform of a vibration detected by the vibration sensor matches a discharge waveform stored in advance.

6. The X-ray generation device according to claim 1,
wherein the discharge phenomenon detection circuit detects the discharge phenomenon based on a change in at least one of a tube current or a tube voltage of the X-ray tube.

7. The X-ray generation device according to claim 1,
wherein the high-voltage generator and the X-ray tube use a neutral point grounding method.

8. The X-ray generation device according to claim 1,
wherein the high-voltage generator and the X-ray tube use an anode grounding method.

9. The X-ray generation device according to claim 1,
wherein the vibration sensor is a displacement sensor or an acceleration sensor.

10. A discharge location identification method for an X-ray generation device including
a high-voltage generator that generates a high voltage,
an X-ray tube that generates X-rays in response to the high voltage supplied from the high-voltage generator,
a discharge phenomenon detection circuit that detects an occurrence of a discharge phenomenon, and
a vibration sensor that is attached to the high-voltage generator, the method comprising:
determining whether the discharge phenomenon occurs in the X-ray tube or the high-voltage generator based on detection results of the discharge phenomenon detection circuit and the vibration sensor.
